(19) United States
(12) Patent Application Publication
(10) Pub. No.: US 2015/0065832 A1
(43) Pub. Date: Mar. 5, 2015

(54) SYSTEM AND METHODS OF IDENTIFYING AND ALERTING A USER OF A HEALTH EVENT

(71) Applicant: Paul Manion, Hoopeston, IL (US)

(72) Inventor: Paul Manion, Hoopeston, IL (US)

(21) Appl. No.: 14/464,246

(22) Filed: Aug. 20, 2014

Related U.S. Application Data
(60) Provisional application No. 61/872,958, filed on Sep. 3, 2013.

(51) Int. Cl. A61B 5/00; A61B 5/1455

(52) U.S. Cl. CPC A61B 5/746; A61B 5/14551; A61B 5/7282; A61B 5/6816; A61B 5/6826; A61B 5/6829; A61B 5/4818; A61B 5/6838; A61B 5/7405; A61B 5/7455; A61B 5/742; A61B 5/0022; A61B 5/7275
USPC 600/340; 600/323

(57) ABSTRACT

A health event monitoring system includes a measurement device that measures a health aspect of a monitored person to provide a measured health aspect, a notification device that provides a notification to a user of the system, and an analyzer that compares the measured health aspect with a predetermined health aspect threshold and which activates the notification device when the measured health aspect passes the predetermined health aspect threshold.

This application claims priority on, and incorporates by reference herein in its entirety, a provisional patent application, having the same title, filed on Sep. 3, 2013, and given U.S. Patent Application Ser. No. 61/872,958, and listing the same inventor, Paul T. Manion.

### FIELD OF THE INVENTION

The present invention relates generally to a system and methods for monitoring a patient such that a health event may be detected and then notifying the patient or a caretaker about the event.

### BACKGROUND OF THE INVENTION

Certain people have a condition or disorder that increases their risk of a health event. While other people may not have any increased risk of a health event, such people may wish to be prepared for a health event in the absence of risk factors.

For purposes of this application, a “health event” is the occurrence of an incident that typically has negative implications for the patient's health. Some health events are immediately life-threatening. Other health events may be life-threatening if untreated for a period of time. Still other health events are not life-threatening at all.

Examples of a health event include decrease or cessation of breathing (e.g., decrease in oxygen saturation in blood), decrease or stopping of heat beat (e.g., pulse), decrease in blood-glucose level, falling asleep unintentionally, sustaining an injury such as from a trauma or fall, seizure, choking, wheezing, and increase or decrease in blood pressure, which may result in losing consciousness, to name a few. Examples of a condition or disorder that increase the risk of a health event, and which themselves are considered a health event, include sleep apnea, asthma, narcolepsy, diabetes, epilepsy, heart disease, arrhythmias, having a pacemaker, and chronic or periodic low or high blood pressure, to name a few.

For purposes of this application, the present invention is discussed in reference to a system and methods configured to detect and alert a user about a sleep apnea-related health event, but the discussion is merely exemplary. The present invention is applicable to any health event that can be detected using a detection component.

Sleep apnea is a relatively common sleep disorder in which breathing is disrupted for various durations. For purposes of this application, a disruption in breathing is termed a “sleep apnea event”, which is a type of health event. Sleep apnea can affect adults as well as children and is generally considered under diagnosed in the medical field. Several types of sleep apnea exist, including, obstructive, central, and complex. In obstructive sleep apnea, a portion of the airway is physically blocked, for example, from enlarged tonsils or collapsed structural feature s in the throat. In central sleep apnea, the brain signals that control breathing are disrupted or cease for a period of time. Complex sleep apnea is a combination of obstructive sleep apnea and central sleep apnea in which a physical obstruction interrupts proper berating and the signals sent by the brain are not functioning properly.

Pauses in breathing caused by sleep apnea may range in duration from ten seconds to several minutes. Sleep apnea may be characterized as “mild” (e.g., patient experiences five to fifteen sleep apnea events per hour of sleep), “moderate” (e.g., patient experiences fifteen to twenty-nine sleep apnea events per hour of sleep), or “severe” (e.g., patient experiences thirty or more sleep apnea events per hour of sleep). Individuals with sleep apnea may not realize that they are having difficulty breathing, even after waking up. Instead, the condition or its symptoms sometimes are first observed by others.

Patients that have sleep apnea may exhibit a number of symptoms, which range in severity. Possible symptoms, other than pauses in breathing, include fatigue during daytime, snoring, impaired alertness, increased reaction time, and vision problems. Over time, these symptoms may cause a number of other more sever health problems such as high blood pressure, heart failure, stroke, diabetes, or depression. These results occur because the pauses in breathing may cause insufficient oxygen to reach the brain for certain periods of time. While sleep apnea can affect anyone, some common risk factors include: being male, relatively high body mass index, age, large neck size, large tonsils, family history of sleep apnea, experiencing gastro-esophageal reflux, and the existence of a nasal obstruction.

Sleep apnea is typically diagnosed by monitoring the oxygen (O₂) concentration in the patient's blood, a process called oximetry. Oximetry may be performed in a patient's residence over night. While sleep apnea also may be diagnosed using polysomnography—that is, a formal sleep study which typically occurs in a sleep laboratory—, home oximetry is generally preferred since it is less expensive.

When a patient is diagnosed with sleep apnea, a health care professional may recommend that the patient sleep with a continuous positive airway pressure (CPAP) device, which is configured to keep a patient's upper airway passages open, and accordingly, maintain regular breathing while the patient is sleeping. A CPAP device may include a mask to be worn over the patient's mouth and nose, an air pump (often in a housing) configured to pump air to the patient through the mask, a humidifier, a power supply or power connector, a hose configured to carry the air from the air pump to the mask, and a carrying case for all of the pieces. Even the most compact configurations of such devices typically weight between 5 lbs and 8 lbs and may have dimensions of between 5″×5″×5″ to 11″×6.7″×5.5″. Such devices may be uncomfortable to use while sleeping, unwieldy to carry around when traveling, and cause generally continuous noise disruption for the patient and any bed partner throughout the night (or other sleeping period, e.g., nap or daytime sleeping).

Another type of treatment for sleep apnea includes an expiratory positive airway pressure (EPAP) device, which is generally smaller than a CPAP device. Such EPAP devices are configured to be positioned over each nostril before sleep and often include a valve that permits more air to move into the airway, but decrease the ease with which air may be exhaled. Accordingly, as a result of the EPAP device, more pressure builds up in the airway, which promotes maintaining the airway in an open position. While such EPEP devices are smaller and easier to transport during traveling, they are generally configured only for single-use and may not be sufficient treatment for those with certain symptoms of sleep apnea.

Yet another device configured to promote maintaining an airway open during sleeping periods is an oral appliance. Such devices may be configured to position the jaw generally forward to maintain the throat in an open position. These devices typically must be personalized to the specific patient and may not be sufficient intervention to maintain regular breathing in patients, especially those with moderate to severe sleep apnea.

Clearly, the available treatment devices are associated with certain limitations and disadvantages.

If the patient no longer wishes to use the available treatment devices or the available treatment devices are ineffective, certain surgical options may decrease or eliminate some or all sleep apnea symptoms. Examples of such surgeries include mouth, tonsil, or throat tissue removal, jaw repositioning, implants positioned in the soft palate of the mouth to promote maintaining a clear airway, nasal surgery (e.g., tracheostomy). Such surgical treatment options are often invasive and expensive.

Clearly, there is a demand for a size-minimized, multiple-use, and non-invasive system and method for identifying a sleep apnea event and alerting a user about the event. The present invention satisfies this demand.

### SUMMARY OF THE INVENTION

Embodiments of the present invention are configured to identify a health event—such as sleep apnea event—and alert a user that such event is occurring or has occurred. In certain embodiments, the present invention includes a detection component, such as a blood oxygen measurement device, and a notification component, such as an audible alarm.

The present invention and its attributes and advantages will be further understood and appreciated with reference to the detailed description below of presently contemplated embodiments, taken in conjunction with the accompanying drawings.

### BRIEF DESCRIPTION OF THE DRAWINGS

FIG. 1 illustrates an embodiment of the system of the present invention including a detection component, management component, and a notification component.

FIG. 2 illustrates an embodiment of the system of the present invention including a detection component, management component, a notification component, stabilization component, storage component, display component, and power supply component.

FIG. 3A illustrates an embodiment of a finger-mounted wearable detection component positioned on a patient's hand.

FIG. 3B illustrates an embodiment of a finger-mounted wearable detection component positioned on a patient's hand with a wrist-mounted stabilization component.

FIG. 3C illustrates an embodiment of a finger-mounted wearable detection component 102C positioned on a patient's hand 103 with a USB plug 105.

FIG. 4 illustrates an embodiment of a finger-mounted wearable detection component positioned on a patient's hand and including a display component.

FIG. 5 illustrates a method embodiment of the present invention.

FIG. 6 illustrates an exemplary computer system that may be used to implement the method according to the invention.

FIG. 7 illustrates an example schematic of a cloud computing system 400 that may be used to implement method according to the present invention.

### DETAILED DESCRIPTION OF THE INVENTION

Embodiments of the present invention are configured to identify a health event—such as sleep apnea event—and alert a user that such event is occurring or has occurred. In certain embodiments, the present invention includes a detection component and a notification component.

A detection component may be any unit configured to identify a health event. A detection component may be configured to measure some symptom or otherwise sense some information about the patient. For purposes of this application, the information collected by a detection component is termed “measured patient information”, such as a “measured blood oxygen level” for example.

An example of a detection component is an oxygen sensor component (e.g., an oximeter or other such device) configured to measure oxygen saturation (SpO₂) of arterial hemoglobin and possibly pulse. An oxygen sensor component may be configured to be wearable on a body site, for example, on a finger, thumb, earlobe, foot, chest, forehead, or other area of the patient's body. In one example embodiment, the invention may be utilized on a narrow area of a person's body so that light may pass through the body part for detection by the invention, such as the person's finger or earlobe, for example. In certain embodiments, the oxygen sensor includes a light emission element and a photo detector element. In such embodiments, the light emission element directs two wavelengths of light through the body site, and the photo detector receives and measures the absorbance of each wavelength of light. A light emission element may be a light emitting diode (LED) in certain embodiments. The absorbance measurement permits calculating the arterial blood in conduits (e.g., vessels or arteries) near the body site, and thereby allows calculation of the blood oxygen level in the user of the person wearing the device.

In addition, embodiments of a detection component maybe configured to be as ergonomic as possible, for example, to facilitate sleeping or doing other activities while wearing it. Such embodiments may come in child sizes, adult sizes, or “small”, “medium”, and “large” sizes. Certain embodiments may be personalized—that is, sized and shaped—for a specific patient.

In certain embodiments, a detection component is configured as a camera unit configured to capture a photograph and/or video—which, for purposes of this application, are collectively referred to as an “image”—of a patient. The image may be analyzed (either generally in real-time after an image is captured or at a later time-delay) to assess the breathing movements or pulse or oxygen saturation of the patient. Examples of such a camera unit may include a webcam, camera embedded in a smart phone or other smart hand-held computing device, standalone digital camera, or other such photographic device.

Certain embodiments of a detection component may include a sound detection component, such as a microphone, positioned and configured to detect exhaling breath of a patient. Such sound detection components may be configured to measure a breathing pattern and detect a sleep apnea event upon departure from the breathing pattern. Also, a patient may make certain distinct noises during a sleep apnea event. Breathing noises may be recorded and an algorithm may be applied to categorize the noise and either indicative or not indicative of a sleep apnea event. The embodiments that utilize a sound detection component can be calibrated by recording distinctive noises that indicate a sleep apnea event. The management component, described in more detail below, may map the noise and compare subsequent similar noises to the calibrated noises.

Another type of detection component may include a motion detection component. Such embodiments may be attached to the patient's body, e.g., abdomen, to measure breathing. Certain embodiments of a detection component may collect pressure information as the patient's abdomen expands and contracts with breathing and exhaling. An algorithm may be run on the measured patient information to identify a breathing pattern and upon departure from the pattern determines that a sleep apnea event is occurring. Accordingly, such embodiments of the invention may utilize pressure or a motion detection component to identify a sleep apnea event.

A detection component may be paired with a stabilization component. A stabilization component is configured to support the position of the detection component. A stabilization component may be especially useful in embodiments intended to be used or worn for longer periods to time, e.g., while sleeping, compared to just taking a short term measurement and removing the detection component. In certain embodiments, the detection component is a finger-mounted detection component and is paired with a stabilization component configured as a wrap band positioned around the outside of the detection component, a patient's wrist, or a patient's finger. If a wrap band is not positioned directly adjacent to or in direct contact with the detection component, a linking component, such as a string, rope, or tie, also may be used to connect the wrap band to the detection component. A wrap band may be configured to cover a part of the detection component, an entire detection component, or a part or portion of a housing of a detection component. (A housing of a detection component may also be the housing of a larger set of components, e.g., a computer system such as a smart phone.)

In certain embodiments, a stabilization component may be configured as a receptacle component configured to retain the detection component in a position to detect information about the patient without being in physical contact with the patient. For example, a receptacle component positioned on a nightstand or dresser maybe configured to position a camera unit-embodiment to face the patient while the patient sleeps, sits, or lies down in bed.

Certain embodiments of the present invention include a management component. A management component may be configured to send and receive information to and from the detection component and other components of the system. The management component may communicate via wireless connection (e.g., WiFi, Bluetooth, RFID, ZigBee, or other known such devices) or wire-based connections (Ethernet, cable, USB cable, micro-USB cable, HDMI, VGA, Jack Plug Audio connector, or others). Other embodiments of the present invention include a physical connection, with a minimal amount of wire between them. For example, a detection element may include a USB plug configured to plug directly into a USB port.

The management component may control when and how the detection component takes measurements of the patient. The management component may process and compute the measured patient information received from the detection component.

When the measured patient information or information calculated from the measured patient information includes trigger information designated as indicating a health event, the measured component may send a command to activate another component—that is, a notification component. A notification component may be configured to alert a system user that a health event has occurred to alert the patient about the event, while in other embodiments, the notification component is configured to alert a health care provider, relative, neighbor, or other person having an interest in the patient's health. A notification component may be configured to produce what is termed an “output” for purposes of this application. For example, the notification component may be configured to emit a sound (e.g., an alarm, song, tone, etc.), cause a movement (e.g., vibration), release a harmless level electric shock, switch on an external device (e.g., television, computer monitor, speakers, headphones, a clock radio, ear-mounted Bluetooth receiver, etc.), turn on a light (either from a light connected to it or by communicating with a remote light system), make a telephone call to a hospital or emergency center, cause a certain screen display, or dispatch an electronic message (e.g., email, text message, push notification, instant message, or other electronic message known in the art).

Trigger information—or that information which triggers the notification element to produce an output configured to alert a system user of a health event—may include a heartbeat (pulse) threshold, breathing rate threshold, or oxygen saturation threshold, for example. In one example, if the patient's blood oxygen saturation is detected to be below a selected threshold (e.g., below 90% or 92% saturation), the notification component may cause an alarm to sound or a vibrator to vibrate. Such alarm may wake up the patient and, in waking up, cause the patient to resume breathing a more normal rate. Alternatively, an alarm may wake a co-sleeping partner who, upon realizing the alarm means that the patient is experiencing a health event, may nudge or otherwise attempt to awaken the patient. Of course, if the patient is not responsive or clearly needs additional medical help the partner can administer such medical help or call a health care provider to do so.

In addition, an alarm may cease emitting the output in several ways: after the elapse of a specified amount of time, after a certain number of alarm events, e.g., rings or buzzes, or upon a detection that the patient's blood oxygen level has returned to a normal level, for example. Each of these methods to stop the alarm can be alternatively or collectively programmed into the management component.

Certain embodiments of the present invention also may include a storage component configured to permit storing measured patient information and other information. Examples of a storage component include a main memory, secondary memory, index, catalog, spreadsheet, or database.

The stored information can be detected at any time, thereby resetting the device to its original condition. The data can also be stored or transferred to another computer or second storage component for purposes of review by a physician, nurse, other health care provider, or insurance company. The management component may contain a removable storage medium, such as a small memory card. An example is a SD/MMC Ms/Pro card. Advantageously, tracking such information may permit health care providers to ascertain certain trends in the patient's health events, and possibly, improve the patient's treatment plan.

Certain embodiments of the present invention include a power supply component. A power supply component may include a battery, electric outlet plug, or other element known in the art to permit storing or transmitting power to the other components of the system. Certain embodiments include multiple power supply components to permit using one as a back-up in case another one fails.

Certain embodiments of the present invention include a user interface configured to be displayed via an input/output display interface such as a display component. A display component may include a touch screen, monitor, or other component known in the art.

Clearly, certain embodiments of the present invention are directed to a system and method for identifying the onset of sleep apnea events and triggering an alarm to wake the patient. In addition, certain embodiment are directed to easily-portable, size-minimized, self-contained monitoring devices. In addition, certain embodiments of the present invention are directed to monitoring devices configured not to disrupt the sleep of the patient's sleeping partner.

The preferred embodiments of the invention will be described in conjunction with the appended drawings provided to illustrate and not to limit the invention, where like designations denote like elements.

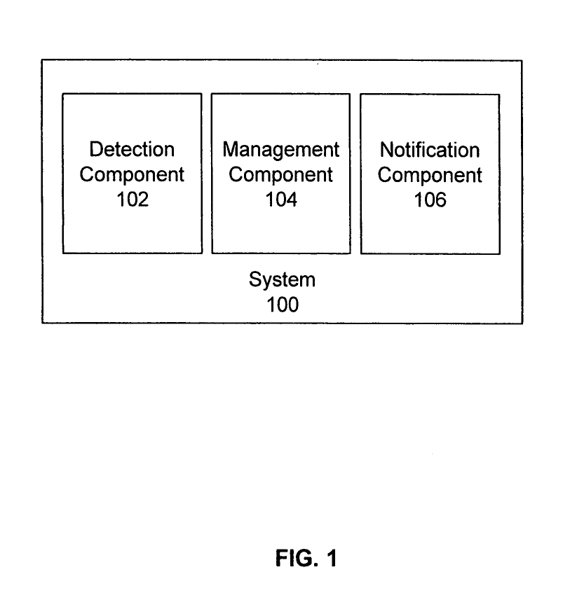

FIG. 1 is a schematic drawing of one example embodiment of the invention 100 that illustrates the present invention including a detection component 102, management component 104, and a notification component 106.

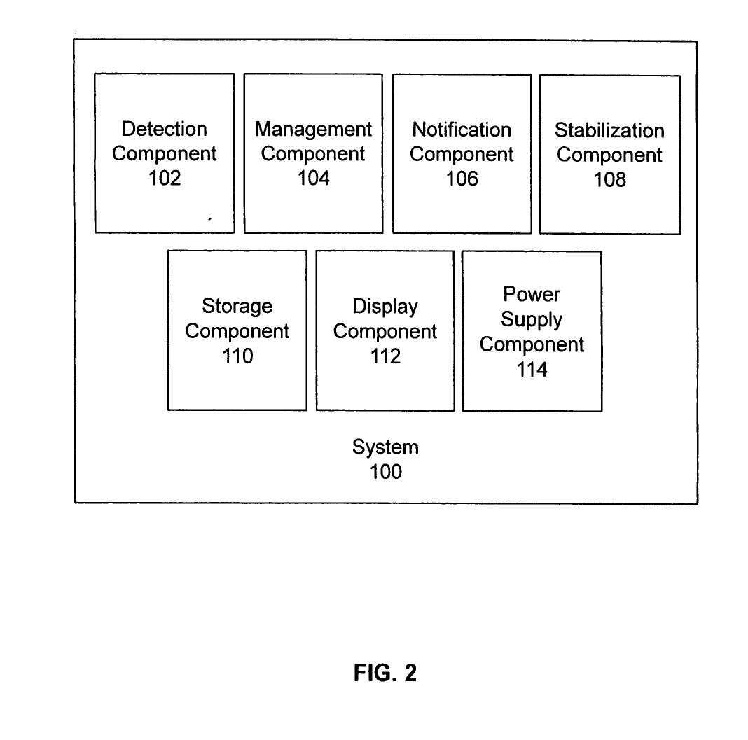

FIG. 2 is a schematic drawing of one example embodiment of the invention 100 that illustrates the present invention including a detection component 102, management component 104, a notification component 106, stabilization component 108, storage component 110, display component 112, and power supply component 114.

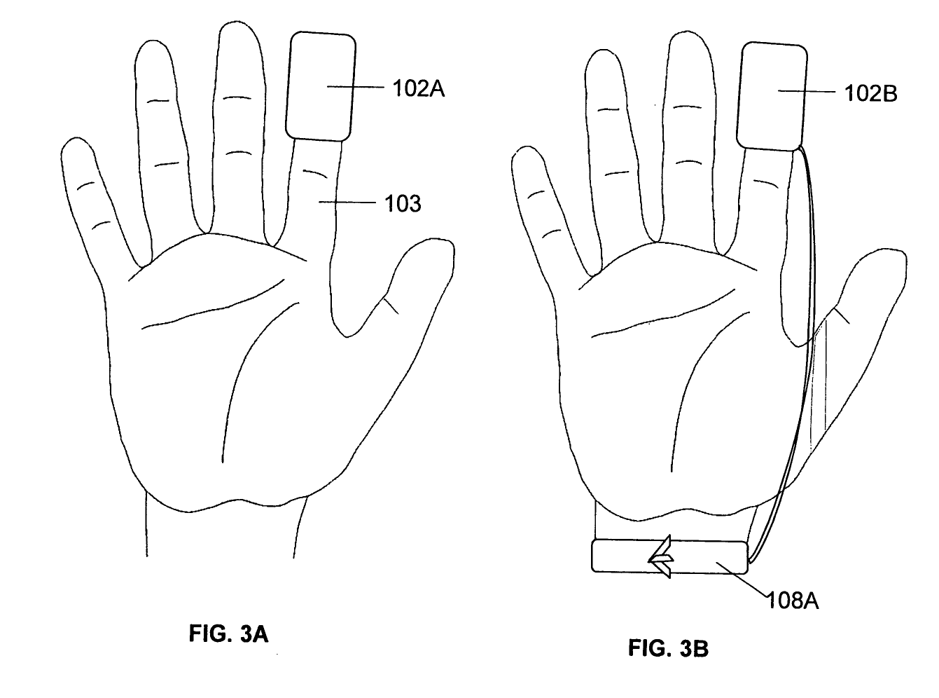

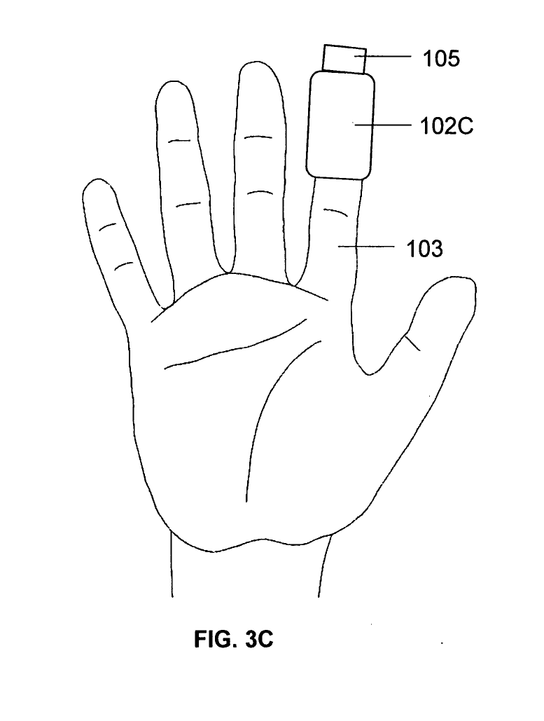

FIG. 3A illustrates an example embodiment of a finger-mounted wearable detection component 102A positioned on a patient's hand 103.

FIG. 3B illustrates an example embodiment of a finger-mounted wearable detection component 102B positioned on a patient's hand 103 with a wrist-mounted stabilization component 108A.

FIG. 3C illustrates an example embodiment of a finger-mounted wearable detection component 102C positioned on a patient's hand 103 with a communication device 105, such as a USB plug.

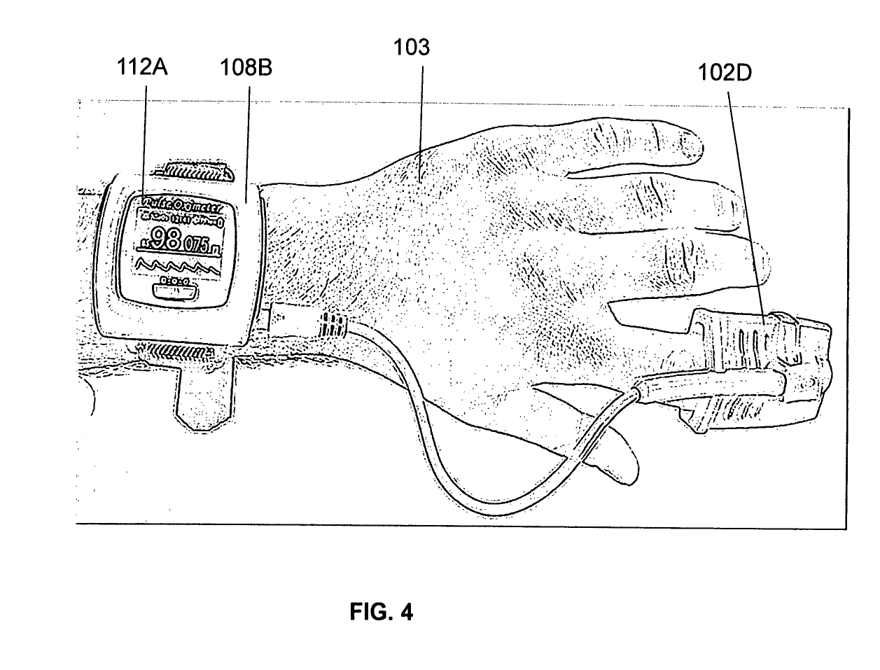

FIG. 4 illustrates an example embodiment of a finger-mounted wearable detection component 102B positioned on a patient's hand 103. In addition, the illustrated wrist-mounted stabilization component 108B includes a display component 112A.

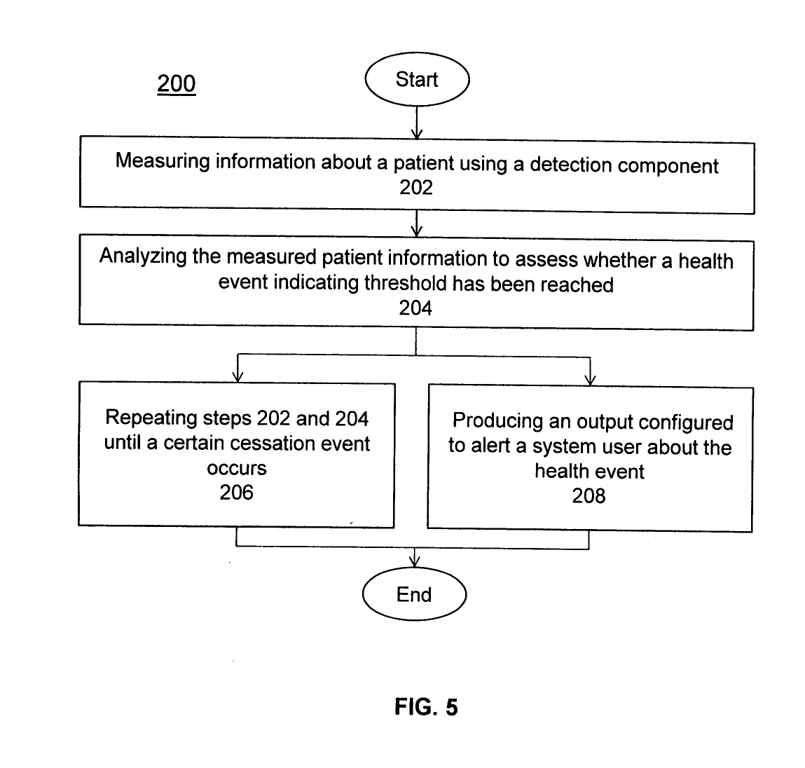

FIG. 5 illustrates an example method embodiment 200 of the present invention. A method embodiment 200 of the present invention may include measuring information about a patient using a detection component 202 and analyzing the measured patient information to assess whether a health event indicating threshold has been reached 204. If the measured patient information does not indicate that a health event is occurring, the system may repeat steps 202 and 204 until a certain cessation event occurs 206. Examples of a cessation event includes loss of power (e.g., the power is switched off), receiving a direction to cease taking measurements from the management component, or other event programmed into the system. If the measured patient information indicates that a health event is occurring or has occurred, the system may produce an output configured to alert a system user about the health event 208.

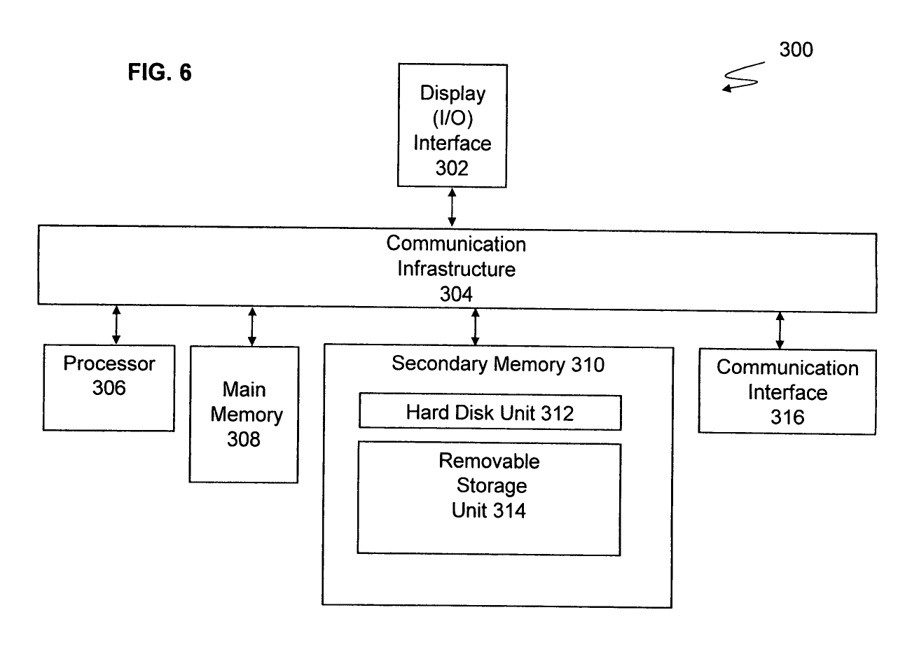

FIG. 6 illustrates an exemplary computer system 300 that may be used to implement the method according to the invention. One or more computer systems 300 may carry out the method presented herein as computer code.

Computer system 300 includes an input/output display interface 302 connected to communication infrastructure 304—such as a bus—, which forwards data such as graphics, text, and information, from the communication infrastructure 304 or from a frame bugger (not shown) to other components of the computer system 300. The input/output display interface 302 may be, for example, a keyboard, touch screen, joystick, trackball, mouse, monitor, speaker, printer, microphone, projector, Google Glass® unit, accelerometer, global positioning system, any other computer peripheral deice, or any combination thereof, capable of measuring, entering, and/or viewing data.

Computer system 300 includes one or more processors 306, which may be a special purpose or a general-purpose digital signal processor that processes certain information. Computer system 300 also includes a main memory 308, for example random access memory (“RAM”), read-only memory (“ROM”), mass storage device, or any combination thereof. Computer system 300 may also include a secondary memory 310 such as a hard disk unit 312, a removable storage unit 314, or any combination thereof. Computer system 300 may also include a communication interface 316, for example, a modem, a network interface (such as an Ethernet card or Ethernet cable), a communication port, a PCMCIA slot and card, wired or wireless systems (such as Wi-Fi, Bluetooth, Infrared), local area networks, wide area networks, intranets, etc.

It is contemplated that the main memory 308, secondary memory 310, communication interface 316, or a combination thereof, function as a computer usable storage medium, otherwise referred to as a computer readable storage medium, to store and/or access computer software including computer instructions. For example, computer programs or other instructions may be loaded into the computer system 300 such as through a removable storage device, for example, a floppy disk, ZIP disks, magnetic tape, portable flash drive, optical disk such as a CD or DVD or Blu-ray, Micro-Electro-Mechanical Systems (“MEMS”), nanotechnological apparatus. Specifically, computer software including computer instructions may be transferred from the removable storage unit 314 or hard disc unit 312 to the secondary memory 310 or through the communication infrastructure 304 to the main memory 308 of the computer system 300.

Communication interface 316 allows software, instructions and data to be transferred between the computer system 300 and external devices or external networks. Software, instructions, and/or data transferred by the communication interface 316 are typically in the form of signals that may be electronic, electromagnetic, optical, or other signals capable of being sent and received by the communication interface 316. Signals may be sent and received using wire or cable, fiber optics, a phone line, a cellular phone link, a Radio Frequency (“RF”) link, wireless link, or other communication channels.

Computer programs, when executed, enable the computer system 300, particularly the processor 306, to implement the method of the invention according to computer software including instructions.

The computer system 300 described herein may perform any one of, or any combination of, these steps of any of the methods presented herein. It is also contemplated that the methods according to the invention may be performed automatically, or may be invoked by some form of manual intervention.

The computer system 300 of FIG. 6 is provided only for purposes of illustration, such that the invention is not limited to this specific embodiment. It is appreciated that a person skilled in the relevant art knows how to program and implement the invention using any computer or like system.

Examples of a computer system 300 include a handheld device and include any small-sized computer device including, for example, a personal digital assistant (“PDA”), smart hand-held computer device, cellular telephone, or a laptop or net book computer, hand held console or MP3 player, tablet, or similar hand held computer device, such as an iPad®, iPad Touch® or iPhone®.

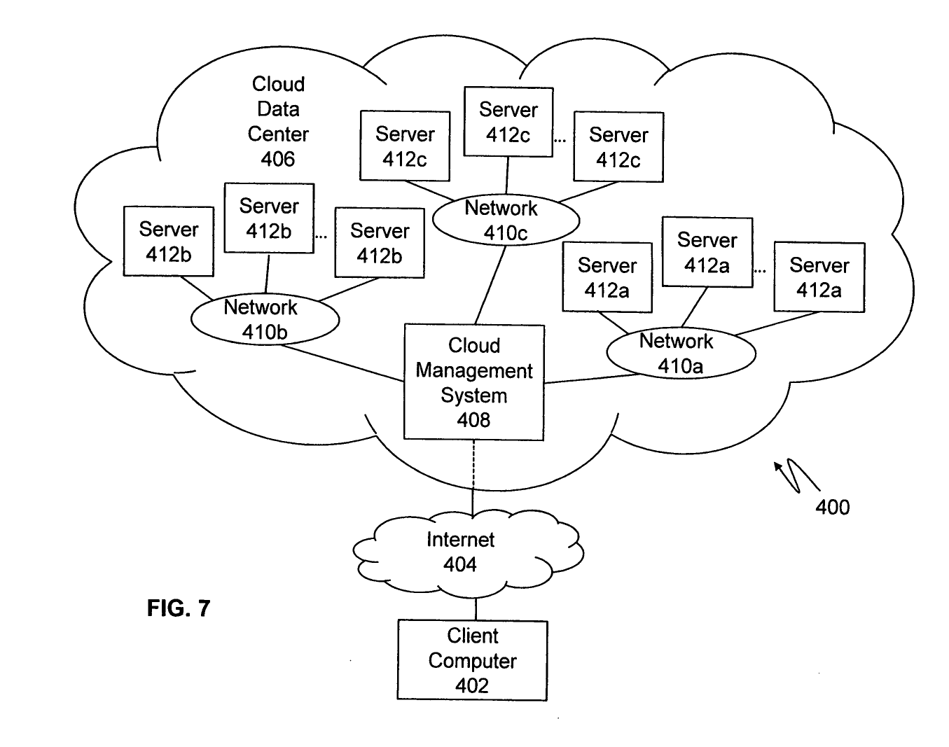

FIG. 7 illustrates an exemplary cloud computer system 400 that may be used to implement the methods according to the present invention. The cloud computing system 400 includes a plurality of interconnected computer environments. The cloud computer system 400 utilizes the resources from various networks as a collective virtual computer, where the services and applications can run independently from a particular computer or service configuration making hardware less important.

Specifically, the cloud computing system 400 includes at least one client computer 402. The client computer 402 may be any device through the use of which a distributed computer environment may be accessed to perform the method disclosed herein, for example, a desktop computer, portable computer, mobile phone, personal digital assistant, tablet to name a few. The client computer 402 includes memory such as random access memory (“RAM”), read-only memory (“ROM”), mass storage device, or any combination thereof. The memory functions as a computer usable storage medium, otherwise referred to as a computer readable storage medium, to store and/or access computer software and/or instructions.

The client computer 402 also includes a communications interface, for example, a modem, a network interface (such as an Ethernet card), a communications port, a PCMCIA slot and card, wired or wireless systems, etc. The communications interface allows communication through transferred signals between the client computer 402 and external devices including networks such as the Internet 404 and cloud data center 406. Communication may be implemented using wireless or wired capability such as cable, fiber optics, a phone line, a cellular phone link, radio waves or other communication channels.

The client computer 402 establishes communication with the Internet 404—specifically to one or more servers—to, in turn, establish communication with one or more cloud data centers 406. A cloud data center 406 includes one or more networks 410a, 410b, 410c managed through a cloud management system 408. Each network 410a, 410b, 410c includes resource servers 412, 412b, 412c, respectively. Servers 412a, 412b, 412c permit access to a collection of computing resources and components that can be invoked to instantiate a virtual machine, process, or other resource for limited or defined duration. For example, one group of resource servers can host and serve an operating system or component thereof to deliver and instantiate a virtual machine. Another group of resource servers can accept requests to host computing cycles or processor time, to supply a defined level of processing power for a virtual machine. A further group of resource servers can host and serve applications to load on an instantiation of a virtual machine, such as an email client, a browser application, a messaging application, or other applications or software. The cloud management system 408 can comprise a dedicated or centralized server and/or other software, hardware, and network tools to communicate with one or more networks 410a, 410b, 410c, such as the Internet or other public or private network, with all sets of resource servers 412a, 412b, 412c. The cloud management system 408 may be configured to query and identify the computing resources and components managed by the set of resource servers 412a, 412b, 412c needed and available for use in the cloud data center 406. Specifically, the cloud management system 408 may be configured to identify the hardware resources and components such as type and amount of processing power, type and amount of memory, type and amount of storage, type and amount of network bandwidth and the like, of the set of resource servers 412a, 412b, 412c needed and available for use in the cloud data center 406. Likewise, the cloud management system 408 can be configured to identify the software resources and components, such as type of Operating System (“OS”), application programs, and the like, of the set of resource servers 412a, 412b, 412c needed and available for use in the cloud data center 406.

The present invention is also directed to computer products, otherwise referred to as computer program products, to provide software to the cloud computing system 400. Computer products store software on any computer useable medium, known now or in the future. Such software, when executed, may implement the methods according to certain embodiments of the invention. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, optical storage devices, Micro-Electro-Mechanical Systems (“MEMS”), nanotechnological storage device, etc.), and communication mediums (e.g., wired and wireless communications networks, local area networks, wide area networks, intranets, etc.). It is to be appreciated that the embodiments described herein may be implemented using software, hardware, firmware, or combinations thereof.

The cloud computing system 400 of FIG. 7 is provided only for purposes of illustration and does not limit the invention to this specific embodiment. It is appreciated that a person skilled in the relevant art knows how to program and implement the invention using any computer system or network architecture.

In one example embodiment the invention includes a system for activating an alarm when a blood oxygen content in a monitored person drops below a predetermined level. The invention includes an oximeter that measures a blood oxygen level in a monitored person to provide a measured blood oxygen level, an analyzer that determines when said measured blood oxygen level falls below a predetermined blood oxygen level, and a notification component activated by the analyzer to provide a notification when the measured blood oxygen level falls below the predetermined blood oxygen level. The system may be releasably secured on a small appendage of the monitored person, the small appendage chosen from the group consisting of an earlobe, a finger, and a toe. The monitored person may be chosen from the group consisting of an adult monitored for sleep apnea and an infant monitored for SIDS. The system may include a clamping device to releasably clamp the oximeter to the monitored person.

The oximeter may be the only measurement device of said system. In other words, the device may allow notification of a user or another predetermined individual or system when a blood oxygen level in the monitored person falls below a predetermined level, without any other health indications. In other words, the notification device may activate upon the single health event of a low blood oxygen saturation level, without measuring other bodily functions such as pulse, heart rate, breathing abnormalities, or the like. Accordingly the system of the current invention may be manufactured in a small size compared to prior art devices, and for a cost below the cost of prior art devices that include multiple measurement or monitoring devices. The simplicity of this embodiment of the invention allows for ease of use by a user in their home. The small size of the device also allows for ease of use with a small child or infant, which may be useful for monitoring and preventing SIDS (sudden infant death syndrome) due to low blood oxygen content, such as during an absence of breathing, in infants and young children.

In one example embodiment the system may continuously monitor blood oxygen level in the monitored person such that the measured blood oxygen level is continuously updated in real time. The notification component may be chosen from an audible notification component that emits a sound, a movement notification component that causes a movement, a shock notification component that releases an electric shock, a switch notification component that switches an external device, an optical notification component that activates a light, a contact notification component that causes contact with a predetermined entity, and a display notification component that displays a notification signal.

An example method of the invention of activating an alarm when a blood oxygen content in a monitored person drops below a predetermined level may include measuring a blood oxygen level in a monitored person to provide a measured blood oxygen level, determining when the measured blood oxygen level falls below a predetermined blood oxygen level, and providing a notification when the measured blood oxygen level falls below the predetermined blood oxygen level. The measuring may be conducted with an oximeter releasably secured to the monitored person on a small appendage of the monitored person, whereby the small appendage may be chosen from the group consisting of an earlobe, a finger, and a toe. Providing the notification may be conducted in response only to the measuring said blood oxygen level and in an absence of all other measurements of bodily activity. The monitored person may be chosen from the group consisting of an adult monitored for sleep apnea and an infant monitored for SIDS. The oximeter may include a clamping device to releasably clamp the oximeter to the monitored person. Measuring the blood oxygen level may be the only measurement conducted in the method, meaning that no other measurement is required to cross a predetermined threshold for the notification component to be activated. Accordingly, the method may include providing a notification only upon the measured blood oxygen level falling below the predetermined blood oxygen level and in an absence of measurement of other bodily events occurring in the monitored person. In such a method the oximeter may continuously monitor the blood oxygen level in the monitored person such that the measured blood oxygen level is continuously updated in real time. The notification may be chosen from an audible notification that emits a sound, a movement notification that causes a movement, a shock notification that releases an electric shock, a switch notification that switches an external device, an optical notification that activates a light, a contact notification that causes contact with a predetermined entity, and a display notification that displays a notification signal.

In another embodiment a health event monitoring system may include a measurement device that measures a health aspect of a monitored person to provide a measured health aspect, a notification device that provides a notification to a user of said system, and an analyzer that compares the measured health aspect with a predetermined health aspect threshold and which activates the notification device when the measured health aspect passes the predetermined health aspect threshold. In such a system the analyzer may activate the notification device in response to measurement of only a single health aspect of the monitored person. The monitored person may be chosen from the group consisting of an adult monitored for sleep apnea and an infant monitored for SIDS. The notification to said user in such a system may comprise a notification that occurs at the site of the measurement device. In other words, the notification occurs at the site of the monitored person, such as waking a monitored sleeping person in their home, as opposed to a device used in a hospital setting wherein a notification event may occur at a remote location to alert hospital personnel that an adverse health event is occurring in a hospital patient under their care. Accordingly, the device of the present invention is suitable in size, price, and notification capabilities for use by a person to monitor themselves or their infant child, for example, in their own home, without intervention by medical personnel.

While the disclosure is susceptible to various modifications and alternative forms, specific exemplary embodiments of the present invention have been shown by way of example in the drawings and have been described in detail. It should be understood, however, that there is not intent to limit the disclosure to the particular embodiments disclosed, but on the contrary, the intention is to cover all modifications, equivalents, and alternatives falling within the scope of the disclosure as defined by the appended claims.

What is claimed is:

1. A system for activating an alarm when a blood oxygen content in a monitored person drops below a predetermined level, comprising:
an oximeter that measures a blood oxygen level in a monitored person to provide a measured blood oxygen level;
an analyzer that determines when said measured blood oxygen level falls below a predetermined blood oxygen level; and
a notification component activated by said analyzer to provide a notification when said measured blood oxygen level falls below said predetermined blood oxygen level.

2. The system of claim 1 wherein said oximeter is releasably secured on a small appendage of the monitored person, said small appendage chosen from the group consisting of an earlobe, a finger, and a toe.

3. The system of claim 1 wherein said monitored person is chosen from the group consisting of an adult monitored for sleep apnea and an infant monitored for SIDS.

4. The system of claim 1 wherein said oximeter comprises a clamping device to releasably clamp said oximeter to said monitored person.

5. The system of claim 1 wherein said oximeter comprises the only measurement device of said system.

6. The system of claim 1 wherein said analyzer activates said notification component only upon said measured blood oxygen level falling below said predetermined blood oxygen level and in an absence of measurement of other bodily events occurring in said monitored person.

7. The system of claim 1 wherein said oximeter continuously monitors said blood oxygen level in said monitored person such that said measured blood oxygen level is continuously updated in real time.

8. The system of claim 1 wherein said notification component is chosen from an audible notification component that emits a sound, a movement notification component that causes a movement, a shock notification component that releases an electric shock, a switch notification component that switches an external device, an optical notification component that activates a light, a contact notification component that causes contact with a predetermined entity, and a display notification component that displays a notification signal.

9. A method of activating an alarm when a blood oxygen content in a monitored person drops below a predetermined level, comprising:
measuring a blood oxygen level in a monitored person to provide a measured blood oxygen level;
determining when said measured blood oxygen level falls below a predetermined blood oxygen level; and
providing a notification when said measured blood oxygen level falls below said predetermined blood oxygen level.

10. The method of claim 9 wherein said measuring is conducted with an oximeter releasably secured to said monitored person on a small appendage of the monitored person, said small appendage chosen from the group consisting of an earlobe, a finger, and a toe.

11. The method of claim 9 wherein said providing said notification is conducted in response only to said measuring said blood oxygen level and in an absence of all other measurements of bodily activity.

12. The method of claim 9 wherein said monitored person is chosen from the group consisting of an adult monitored for sleep apnea and an infant monitored for SIDS.

13. The method of claim 10 wherein said oximeter comprises a clamping device to releasably clamp said oximeter to said monitored person.

14. The method of claim 9 wherein said measuring said blood oxygen level comprises the only measurement conducted in said method.

15. The method of claim 9 wherein said providing a notification occurs only upon said measured blood oxygen level falling below said predetermined blood oxygen level and in an absence of measurement of other bodily events occurring in said monitored person.

16. The method of claim 9 wherein said oximeter continuously monitors said blood oxygen level in said monitored person such that said measured blood oxygen level is continuously updated in real time.

17. The method of claim 9 wherein said notification is chosen from an audible notification that emits a sound, a movement notification that causes a movement, a shock notification that releases an electric shock, a switch notification that switches an external device, an optical notification that activates a light, a contact notification that causes contact with a predetermined entity, and a display notification that displays a notification signal.

18. A health event monitoring system, comprising:
a measurement device that measures a health aspect of a monitored person to provide a measured health aspect;
a notification device that provides a notification to a user of said system; and
an analyzer that compares said measured health aspect with a predetermined health aspect threshold and which activates said notification device when said measured health aspect passes said predetermined health aspect threshold.

19. The system of claim 18 wherein said analyzer activates said notification device in response to measurement of only a single health aspect of said monitored person and wherein said notification to said user comprises a notification that occurs at the site of the measurement device.

20. The system of claim 18 wherein said monitored person is chosen from the group consisting of an adult monitored for sleep apnea and an infant monitored for SIDS.
